(19) United States
(12) Patent Application Publication
(10) Pub. No.: US 2008/0269334 A1
(43) Pub. Date: Oct. 30, 2008

(54) Highly Concentrated Pourable Aqueous Solutions Of Potassium Ibuprofen, Their Preparation And Their Use

(75) Inventors: Partick C. Hu, Baton Rouge, LA (US); Gregory H. Lambeth, Baton Rouge, LA (US); Arcelio J. Malcolm, Baton Rouge, LA (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 12/066,371

(22) PCT Filed: Sep. 15, 2006
(86) PCT No.: PCT/US06/35932

§ 371 Date: Mar. 10, 2008

Related U.S. Application Data
(60) Provisional application No. 60/719,018, filed on Sep. 19, 2005.

(51) Int. Cl. A61K 31/192; A61P 29/00

(52) U.S. Cl. 514/570

(57) ABSTRACT

Concentrated pourable potassium ibuprofen liquid compositions, and preparation and uses thereof are described. They are composed of (i) at least 60 wt % of potassium ibuprofen in dissolved form; (ii) water; (iii) one or more polyethylene glycols, (iv) optionally up to 5 wt % of at least one C₂₋₃ alkanol; and (v) optionally, ibuprofen in free acid form. The weight ratio of (ii):(iii) is at least 0.9:1. Compositions containing as much as 85.2 wt % of potassium ibuprofen in dissolved form have been successfully made. The highest level of potassium ibuprofen in dissolved form achieved in experiments reported in the presently-known prior art is but 24.2 wt %. The present compositions are suitable for use in the preparation of pharmaceutical products and various pharmaceutical dosage forms such as liquid-filled soft gelatin capsules, syrups, elixirs, suspensions; solid dosage forms such as tablets or caplets; and topically-applied products. such as lotions, creams or ointments.

### TECHNICAL FIELD

This invention relates to new highly concentrated aqueous liquid compositions of what are deemed to be at least partially solvated and/or at least partially ionized and/or at least complexed potassium 2-(4-isobutylphenyl)propionate. Thus it is believed that the compositions may contain at least some potassium cations and 2-(4-isobutylphenyl)-propionate anions. If one were to remove all of the liquid from such compositions, the resultant solids would comprise at least a predominate amount of potassium 2-(4-isobutylphenyl)propionate (often referred to herein as “potassium ibuprofen”).

Consequently for convenience, and convenience only, the term “dissolved” in connection with potassium ibuprofen is often used in this document. This term is used in its ordinary meaning to specify that a quantity of potassium ibuprofen has been caused to pass into solution. This term is not intended as a representation that what is in the liquid composition is actually potassium 2-(4-isobutylphenyl)propionate as such. Rather, the term is used to specify that upon the removal of all of the liquid from the concentrated liquid composition, potassium 2-(4-isobutylphenyl)propionate would be present in a solids phase, and that while in the concentrated liquid composition it may be partially or entirely in solvated form and/or in ionized form and/or in complexed form and/or in some other dissolved or soluble form. In short, the amount of the potassium ibuprofen which has dissolved is not in the form of a visually perceivable separate phase such as a solid phase or a gel phase.

### BACKGROUND

U.S. Pat. Nos. 4,859,704 and 4,861,797 describe the formation and use of certain liquid ibuprofen compositions in which there are approximately 25 mg to 400 mg of the ibuprofen composition per 5 mL of the aqueous solution. These values correspond to 0.5 to 8 wt % solutions. A methylcellulose composition such as sodium carboxymethylcellulose with or without sucrose is used as a component in these solutions. According to the latter patent, “the methylcellulose composition renders the ibuprofen soluble in the aqueous medium”.

U.S. Pat. Nos. 5,071,643 and 5,360,615 describe solvent systems for enhancing the solubility of acidic, basic, or amphoteric pharmaceutical agents indicated to be suitable for filling soft gelatine capsules. Among the pharmaceuticals dealt with in the patents is ibuprofen. The patents report in Example III and Table 2 that the solubility of ibuprofen in a solvent system composed of 402 mg of ibuprofen, 100 mg of polyethylene glycol 600, 19.8 mg (3.3 wt %) of glycerin or propylene glycol, 38.4 mg (6.4 wt %) of water, and 38.4 mg of potassium hydroxide (0.3 mole equivalents of hydroxide per mole of ibuprofen) was 67%. Thus the amount of potassium ibuprofen in this solution was at best only 23.9%. The other systems reported in the patent in which ibuprofen was used resulted in even lower amounts of dissolved potassium ibuprofen. The patent notes that the presence of glycerin, propylene glycol or polyvinylpyrrolidone enhances the solubility of acidic pharmaceutical agents in such solutions.

U.S. Pat. No. 5,912,011 discloses compositions in which ibuprofen is dissolved in a polyoxyethylene sorbitan fatty acid ester and treated with KOH. From the proportions given in each of claims 1 and 5 of that patent, the maximum theoretical concentration of dissolved potassium ibuprofen would be 48.4 wt %, in a system containing 48 wt % of the fatty acid ester and 3.6 wt % of water. In the experimental data presented in that patent (Table 1), the concentration of dissolved potassium ibuprofen was 24.2 wt % in a system also composed of 35.2 wt % of ibuprofen, 33.3 wt % of polyethylene glycol (PEG 600), and 7.3 wt % of water.

U.S. Pat. No. 6,387,400 describes an indicated way of increasing the concentration of a pharmaceutically active ingredient such as ibuprofen in solutions useful in dosage forms such as in soft gelatin capsules. The process involves use of polyethylene glycol having a molecular weight of about 200 Daltons to about 100,000 Daltons as a solvent for in situ formed sodium or potassium salts of the pharmaceutically active ingredient. While solutions with somewhat higher concentrations are achieved by the process of the patent, unfortunately the maximum concentration of dissolved potassium ibuprofen shown is about 24.2 wt %, and this was achieved where less than about 50 mole % of the ibuprofen was converted to the potassium salt. Also, complete conversion to either sodium or potassium salts of such ingredient was not achieved inasmuch as attempts to push the reaction to higher concentrations resulted in undesirable reaction with the polyethylene glycol solvent. In order to minimize this undesirable side reaction, elaborate processing steps are employed in the process.

Thus despite these prior efforts by those skilled in the art, a need exists for highly concentrated (e.g., 60 wt % and above), stable, pourable liquid potassium ibuprofen compositions that are easy to prepare from ibuprofen on an economical basis, and for effective ways of producing and using such compositions.

### SUMMARY OF THE INVENTION

This invention provides, among other things, highly concentrated, stable, pourable liquid compositions that are easy to prepare from ibuprofen on an economical basis. These concentrated compositions are comprised of potassium ibuprofen dissolved in approximate concentrations of at least about 60 wt %, preferably at least about 70 wt %, more preferably at least about 80 wt %, and still more preferably between about 80 and about 90 wt % in a special solvent system enabling the achievement of such high concentrations. Included in the compositions of this invention are compositions which, because of their high concentrations of dissolved potassium ibuprofen, are eminently suitable for use in the preparation of a variety of pharmaceutical preparations in which ibuprofen or its potassium salt are active ingredients. In addition, this invention also provides preferred compositions which possess viscosities and volatilities that are well-suited for filling pharmaceutically-acceptable capsules such as gelatine capsules.

The solvent systems of the concentrated liquid compositions of this invention are comprised of water and at least one polyethylene glycol. Preferably the solvent system also contains at least one C₂₋₃ alkanol. Mixtures of two or more such alkanols and/or two or more polyethylene glycols can be used. The components of the solvent system used are in proportions that provide pourable highly concentrated solutions (e.g. at least about 60 wt % solutions) of (a) potassium ibuprofen or (b) mixtures of potassium ibuprofen and ibuprofen in free acid form in which the molar ratio of (a):(b) is greater than about 9:1.

One preferred embodiment of this invention is a concentrated pourable liquid composition comprised of (i) about 60 to about 86 wt % (more preferably about 70 to about 86 wt %, and still more preferably about 80 to about 86 wt %) of potassium ibuprofen in dissolved form in a solvent system comprised of (ii) water, (iii) at least one polyethylene glycol, and (iv) at least one C₂₋₃ alkanol; and (v) optionally, ibuprofen in free acid form. The polyethylene glycol(s) used typically have a number average molecular weight in the range of about 200-2000 Daltons, preferably in the range of about 200-800 Daltons, more preferably in the range of about 200-600 Daltons, and still more preferably approximately 400 Daltons. As a group, the most desirable polyethylene glycols for use in this invention are one or more polyethylene glycols having a number average molecular weight of at least about 200 Daltons that is or are in the liquid state at about 20° C. When the composition is devoid of ibuprofen in its free acid state, or when the composition contains ibuprofen in its free acid state in an amount of up to about 4 wt %, the weight ratio of (ii):(iii) will typically be in the range of about 1:1 to about 2:1 and the weight ratio of (ii):(iv) will typically be in the range of about 3 to about 10. In this embodiment, it is particularly desirable to proportion the water, polyethylene glycol and alkanol components of the solvent such that the weight ratio of water:polyethylene glycol:alkanol is in the ranges of about 3.5-8.8:2.0-7.7:1.

Another embodiment of this invention is a concentrated liquid composition comprised of (i) about 78 to about 88 wt % of potassium ibuprofen in dissolved form; (ii) about 5 to about 10 wt % of water; (iii) more than about 1 wt % and less than about 10 wt % (preferably in the range of about 3 to about 9.5 wt %) of one or more polyethylene glycols, (iv) optionally up to about 5 wt % of at least one C₂₋₃ alkanol (preferably ethanol); and (v) optionally up to about 10 wt % of ibuprofen in free acid form; wherein the weight ratio of (ii):(iii) is at least about 0.9:1; and wherein the composition is pourable at least at about 25° C. In this embodiment, preferably:
A) the concentrated liquid composition contains about 80 to about 86 wt % of potassium ibuprofen in dissolved form; and/or
B) the amount of water in such composition is in the range of about 7.5 to about 10 wt %; and/or
C) the amount of polyethylene glycol(s) in such composition is more than about 3 wt % and less than about 10 wt %, and more preferably is at least about 5 wt % but less than about 10 wt %;
D) the number average molecular weight of the polyethylene glycol(s) used in such composition is preferably in the range of about 200-2000 Daltons, more preferably is in the range of about 200-800 Daltons, still more preferably is in the range of about 200-600 Daltons, and even more preferably is about 400; and/or
E) at least one of the optional components (iv) and (v) is present in such composition, and more preferably both of the optional components (iv) and (v) are present in such composition; and/or
F) the amount of ibuprofen in free acid form, when present in such composition, is up to about 4 wt %, more preferably up to about 3 wt % and still more preferably is up to about 2 wt %; and/or
G) the weight ratio of polyethylene glycol(s):ibuprofen in free acid form (if ibuprofen in free acid form is present in such composition) is at least about 2.2:1, and more preferably is at least about 3.2:1; and/or
H) the weight ratio of polyethylene glycol(s)+C₂₋₃ alkanol(s):ibuprofen in free acid form (if ibuprofen in free acid form is present in such composition) is at least about 3:1, and more preferably is at least about 4.8:1; and/or
I) the weight ratio of water:polyethylene glycol(s) in the composition is at least about 1:1 and preferably is in the range of about 1.02-1.80:1; and/or
J) if the weight ratio of water:polyethylene glycol(s) in the composition is below about 1:1, the weight ratio of water:C₂₋₃ alkanol(s) in the composition and the weight ratio of polyethylene glycol(s):C₂₋₃ alkanol(s) in the composition are each, independently, greater than about 6:1, more preferably are each, independently, greater than about 7:1, and still more preferably are each, independently, greater than about 8:1.

The foregoing preferences of A) through J) can be utilized independently of each other, or in combinations of any two or more of them.

Another embodiment of this invention is a process of producing a concentrated potassium ibuprofen composition, which process comprises:
a) forming a mixture of ibuprofen, potassium base, water, and preferably at least one C₂₋₃ alkanol, the ratio of equivalents of potassium base to ibuprofen used being in the range of about 0.9:1 to 1:1 and preferably in the range of about 0.95:1 to about 0.98:1;
b) partially removing alkanol (when used) and water from mixture from a) thereby forming a product mixture enriched in dissolved potassium ibuprofen but not devoid of alkanol (when used) and water;
c) either continuing or discontinuing removal of alkanol (when used) and water as in b), and mixing at least one polyethylene glycol with product mixture from b) to form a pourable polyethylene glycol-containing composition; and
d) if removal of alkanol (when used) and water is discontinued in c), resume removal of alkanol (when used) and water, in this case from pourable polyethylene glycol-containing composition from c) to thereby form a more concentrated pourable potassium ibuprofen liquid composition comprised of (i) potassium ibuprofen; (ii) water; (iii) at least one polyalkylene glycol; (iv) optionally at least one C₂₋₃ alkanol; and (v) optionally ibuprofen in free acid form; and
e) using amounts of ibuprofen, potassium base, water, polyethylene glycol(s) and optionally alkanol(s) in the process and removing amounts of alkanol(s) (when used) and water in the process that provide a composition containing at least about 60 wt % (preferably at least about 70 wt %, more preferably in the range of about 78 to about 88 wt %, and still more preferably in the range of about 80 to about 86 wt %) of potassium ibuprofen in dissolved form.
   Preferably, in c) above, removal of alkanol (when used) and water is discontinued and resumed as in d). However, if desired, the mixing as in c) of at least one polyethylene glycol with product mixture from b) can be conducted while continuing removal of alkanol (when used) and water. In such case, removal of alkanol (when used) and water can be continued after finishing the mixing of at least one polyethylene glycol with product mixture from b) has been completed, when such removal is desired in order to further concentrate the composition and thereby achieve in the composition a desired higher weight percentage of potassium ibuprofen in dissolved form.

In a preferred embodiment of this invention in which an alkanol is used, a process of producing a concentrated potassium ibuprofen composition is a method which comprises:
a) forming a mixture of ibuprofen, potassium base, water, and at least one C₂₋₃ alkanol, the ratio of equivalents of potassium base to ibuprofen used being in the range of about 0.9:1 to 1:1;
b) partially removing alkanol and water from mixture from a) thereby forming a product mixture enriched in dissolved potassium ibuprofen but not devoid of alkanol and water;
c) discontinuing removal as in b, and mixing at least one polyethylene glycol with product mixture from b) to form a pourable polyethylene glycol-containing composition; and
d) resume removal of alkanol and water, in this case from pourable polyethylene glycol-containing composition from c) to thereby form a more concentrated pourable potassium ibuprofen liquid composition comprised of (i) potassium ibuprofen; (ii) water; (iii) at least one polyalkylene glycol; (iv) optionally at least one C₂₋₃ alkanol; and (v) optionally ibuprofen in free acid form; and
e) using amounts of ibuprofen, potassium base, water, polyethylene glycol(s) and alkanol(s) in the process and removing amounts of alkanol(s) and water in the process that provide a composition containing at least about 60 wt % of potassium ibuprofen in dissolved form.
   Desirably, when forming compositions containing in the range of about 78 to about 88 wt % or more preferably in the range of about 80 to about 86 wt % of potassium ibuprofen in dissolved form, the amounts of ibuprofen, potassium base, water, polyethylene glycol(s) and alkanol(s) used in the process and the amounts of C₂₋₃ alkanol(s) and water removed in the process are such that the composition formed complies with at least one (and more desirably, two or more) of the preferences A) through J) as described above. The manner in which adjustment or control of such amounts can be effected, when not already known, can readily be determined by the simple expedient of performing a few experimental runs to determine in any given situation the effect of these variables on the makeup of the composition produced.

In conducting the above process, it is especially preferred to effect the removals as in b) and d) by use of the same or different evaporation procedures such as by distillation, flashing, azeotropic distillation, vacuum distillation, or the like. Such procedure(s) are typically conducted at temperatures of up to about 70-80° C., although any temperature can be used that effects the removals at a satisfactory rate and that does not result in significant color development or decomposition in or of the composition. Of such evaporation procedures, stripping is a particularly preferred procedure, at least when operating on a laboratory scale.

Another process of this invention is as described above except that removal is not discontinued as specified in c). Instead, the removal is continued while introducing and mixing at least one polyethylene glycol with product mixture from b) to form a concentrated pourable polyethylene glycol-containing composition.

Yet another embodiment of this invention is a process of producing a concentrated liquid composition of this invention, which process comprises:
a) forming a mixture of ibuprofen, potassium base, water, and at least one C₂₋₃ alkanol (preferably ethanol);
b) optionally stripping alkanol and water from mixture from a);
c) optionally while continuing stripping alkanol and water as in b), mixing at least one polyethylene glycol (preferably having a number molecular weight in the range of about 200 to about 2000 Daltons, more preferably in the range of about 200 to about 800 Daltons, and still more preferably in the range of about 200 to about 600 Daltons,) with product mixture from b) to form a polyethylene glycol-containing mixture; and
d) optionally continuing to strip alkanol and water from polyethylene-containing mixture from c);
   such process being characterized in that at least one of the optional stripping steps of b), c), or d) is carried out such that there is formed as a product of the process, a concentrated pourable liquid composition comprising (i) potassium ibuprofen in dissolved form; (ii) water; (iii) at least one polyalkylene glycol, (iv) optionally at least one C₂₋₃ alkanol, and optionally ibuprofen in free acid form; and wherein the potassium ibuprofen is in dissolved form and wherein the amount of dissolved potassium ibuprofen is in the range of about 60 to about 90 wt %, wherein the composition contains about 5 to 10 wt % of water and contains more than about 1 wt % (preferably more than about 3 wt % and more preferably at least about 5 wt %) but less than about 10 wt % of at least one polyethylene glycol, and wherein the composition is pourable at least at about 25° C. By suitably proportioning the ratio of equivalents of potassium base to ibuprofen used (e.g., by employing these components in a ratio in the range of about 0.9 to less than about 1 equivalent of potassium base per equivalent of ibuprofen used), the product of the process contains both potassium ibuprofen and ibuprofen in free acid form such that the process directly results in the formation of a product composition of this invention in which the molar ratio of potassium ibuprofen to ibuprofen in free acid form is greater than about 9:1. However, adjustments can be made in the molar ratio of potassium ibuprofen to ibuprofen in free acid form after completion of the process by adding more potassium base to the product formed in the reaction to increase this molar ratio or, conversely, by adding more ibuprofen to the product formed in the reaction to decrease this molar ratio, provided that the additions do not result in precipitate formation. In either such case, the final molar ratio of potassium ibuprofen to ibuprofen in free acid form should be greater than about 9:1.

Another especially preferred embodiment of this invention is a concentrated liquid composition that is pourable at least at about 25° C. comprised of (i) about 78 to about 88 wt % (more desirably in the range of about 80 to about 86 wt %) of potassium ibuprofen in dissolved form; (ii) about 5 to about 10 wt % of water; and (iii) more than about 1 wt % but less than about 10 wt % of one or more polyethylene glycols, (iv) at least one C₂₋₃ alkanol, and (v) ibuprofen in free acid form, wherein:
1) the weight ratio of polyethylene glycol(s):ibuprofen in free acid form is at least about 2.2:1 (more preferably is at least about 3.2:1); or
2) the weight ratio of polyethylene glycol(s): C₂₋₃ alkanol(s):ibuprofen in free acid form is at least about 3:1, (more preferably is at least about 4.8:1); and
3) the weight ratio of water:polyethylene glycol(s) in the composition is at least about 1:1, or if the weight ratio of water:polyethylene glycol(s) in the composition is below about 1:1, the weight ratio of water:C₂₋₃ alkanol(s) in the composition and the weight ratio of polyethylene glycol(s):C₂₋₃ alkanol(s) in the composition are each, independently, greater than about 6:1, more preferably are each, independently, greater than about 7:1, and still more preferably are each, independently, greater than about 8:1.

Still other embodiments and features of this invention will be still further apparent from the ensuing description and appended claims.

### FURTHER DETAILED DESCRIPTION OF THE INVENTION

Despite their high concentrations of potassium ibuprofen, the concentrated liquid compositions of this invention are pourable liquids which can be readily handled and used in processing operations involved in the preparation of pharmaceutical dosage forms. As used herein the term “pourable” means that the liquid composition of this invention, at least when at a temperature of about 25° C., can be caused to flow or fall as from one container to another, or into, over, or on something without application of any special force other than gravity.

Concentrated liquid compositions of this invention in which ethanol of pharmaceutically-acceptable purity for internal human consumption is present, are suitable for use as, or in the manufacture of, pharmaceutical preparations for internal and external usage. On the other hand, compositions of this invention containing one or more of (1) ethanol not of pharmaceutically-acceptable purity for internal human consumption, (2) 1-propanol and (3) 2-propanol, are not intended as such for direct internal human usage. Instead, such compositions are adapted primarily for other uses such as in production of external topical skin preparations or the like. However, liquid compositions of this invention containing one or more of such alcohols (1), (2), and (3) can be converted into pharmaceutical preparations for internal human consumption, or used in the manufacture of pharmaceutical preparations for internal human consumption by ensuring that no ethanol not of pharmaceutically-acceptable purity for internal human consumption, no 1-propanol, and no 2-propanol, other than perhaps a harmless trace amount of any such alcohol (e.g. less than about 0.1 wt %) remains in the finished pharmaceutical product. In other words, any and all such alcohol(s), if present, are to be removed or converted into pharmaceutically-acceptable ingredients for internal human consumption so that the finished pharmaceutical product for internal human consumption does not contain more than a harmless trace amount of any such alcohol(s). Azeotropic distillation with appropriate substances (e.g., benzene, 2,2,4-trimethylpentane, trichloroethylene, or ethyl ether) used as entrainers is a typical known way of efficiently separating water and alcohol from each other.

Typically, the concentrated liquid compositions of this invention as formed are clear single phase liquid composition (in other words, the composition as viewed by the naked eye is a transparent solids-free solution having only one liquid phase). In addition, such clear single phase liquid compositions are typically (and preferably) stable in the sense that they remain visually as a clear single phase composition for at least about 400 hours when stored promptly after preparation in a closed container in the absence of light and at a temperature in the range of about 5 to about 70° C. In order to evaluate the stability of a highly concentrated liquid composition of this invention to see if it meets this preferred criterion, it is not necessary to run tests at various temperatures within the range of about 5 to about 70° C. A suitable test procedure is to place a sample of a clear single phase promptly after preparation in a closed container in the absence of light in a closed container at a constant temperature of about 5° C. for about 400 hours. If promptly after such storage no separate solid or separate liquid phase in the sample can be seen by the naked eye on viewing the composition under light in the visible wavelength range, the sample is deemed to be stable. It is not necessary to perform the same storage tests at any temperature above about 5° C. because if the sample passes the foregoing test at about 5° C., experience has shown that it will pass the test at higher temperatures within the range of about 5 to about 70° C.

Thus concentrated clear single phase liquid compositions of this invention in which the alkanol present is ethanol of pharmaceutically-acceptable purity for internal human consumption (especially when they are also stable compositions as discussed above) are highly suitable for use in the preparation of any of a variety of pharmaceutical preparations in desired dosage forms, such as filled soft capsules, as well as syrups, elixirs, suspensions, and other pharmaceutical dosage forms including solid dosage forms such as tablets, caplets, and filled hard gelatin capsules. Because the single phase liquid compositions of this invention are stable for at least about 400 hours promptly after preparation, such compositions are likely to remain in the same stable condition for even longer periods of time as long as they are not subjected to destructive high temperatures or some other condition adverse to stability.

The concentrated clear single phase liquid compositions of this invention in which the alkanol present is one or more C₂₋₃ alkanols other than ethanol of pharmaceutically-acceptable purity for internal human consumption (especially when they are also stable compositions as discussed above) are highly suitable for use in the preparation of various pharmaceutical dosage forms for external administration, such as salves, creams, ointments, lotions, topically applied liquids, and like forms.

As between (1) the clear single phase liquid compositions of this invention in which the alkanol present is solely ethanol of pharmaceutically-acceptable purity for internal human consumption, especially when the compositions are also stable compositions and (2) the clear single phase liquid compositions of this invention in which at least one C₂₋₃ alkanol is other than ethanol of pharmaceutically-acceptable purity for internal human consumption, especially when the compositions are also stable compositions, the compositions of (1) are preferred because of their wider fields of use.

In some cases the concentrated liquid compositions of this invention as formed may be hazy, cloudy, or turbid in appearance and/or contain particles that can be removed by suitable physical techniques such as vacuum filtration or centrifugation at up to about 80° C., and which nevertheless contain at least about 60 wt % of dissolved potassium ibuprofen. In any case where a slight amount of haze and/or cloudiness persists in the composition, though less preferred, the composition can be use for preparation of pharmaceutical products in desired dosage forms such as products for external human application such as suspensions, salves, creams, and lotions. If the alkanol present in such compositions is solely ethanol of pharmaceutically-acceptable purity, the composition may also be used in the preparation of suspensions for internal human administration, if desired.

Use of any ingredient other than potassium ibuprofen, water, ethanol, 1-propanol, 2-propanol, or a mixture of any two or more of them is not required to produce the concentrated liquid compositions of this invention. Thus components such as carboxymethylcellulose composition and/or sucrose solubilizing agents, propylene glycol, glycerine, or polyoxyethylene sorbitan fatty acid ester and/or propylene glycol are not required. In fact, some of these additives can be undesirable because of their tendency to form esters in liquid compositions containing one or more polyethylene glycols. Especially preferred compositions of this invention are devoid of glycerine, propylene glycol, polyvinylpyrrolidone, and polyoxyethylene sorbitan fatty acid ester.

It will be recalled that a typical process of this invention for producing a concentrated liquid composition of this invention comprises:
a) forming a mixture of ibuprofen, potassium base, water, and at least one C₂₋₃ alkanol, the ratio of equivalents of potassium base to ibuprofen used being in the range of about 0.9:1 to about 1:1;
b) partially removing alkanol and water from mixture from a) thereby forming a product mixture enriched in dissolved potassium ibuprofen but not devoid of alkanol and water;
c) discontinuing removal as in b, and mixing at least one polyethylene glycol with product mixture from b) to form a pourable polyethylene glycol-containing composition; and
d) resume removal of alkanol and water, in this case from pourable polyethylene glycol-containing composition from c) to thereby form a more concentrated pourable potassium ibuprofen liquid composition comprised of (i) potassium ibuprofen; (ii) water; (iii) at least one polyalkylene glycol; (iv) optionally at least one C₂₋₃ alkanol; and (v) optionally ibuprofen in free acid form, wherein the amounts of alkanol and water removed in b) and d), and the amount of polyethylene glycol(s) used in c) provide a composition containing at least about 60 wt % of potassium ibuprofen in dissolved form; in the range of about 5 to about 10 wt % of water; in the range of more than about 1 wt % but less than about 10 wt % of polyethylene glycol(s); optionally up to about 5 wt % of at least one C₂₋₃ alkanol; and (v) optionally up to about 2.3 wt % of ibuprofen in free acid form.

As seen from the above, the processes of this invention for producing the concentrated liquid compositions of this invention involve a step of forming a mixture of ibuprofen, potassium base, water, and preferably at least one C₂₋₃ alkanol, the ratio of equivalents of potassium base to ibuprofen used being in the range of about 0.9:1 to about 1:1. Preferably, some or all of the at least one C₂₋₃ alkanol is added in step a) of the process. This is advantageous in that in addition to increasing the solubility of potassium ibuprofen in water, the C₂₋₃ alkanol(s) used help(s) in controlling the amount of foam formed during the acid-base reaction in a). Also, the presence of at least one water miscible C₂₋₃ alkanol in step a) of the process facilitates dissolution of ibuprofen and thereby expedites the formation of potassium ibuprofen in situ. Moreover, in conducting step a) the addition of at least one C₂₋₃ alkanol in step a) of the process facilitates agitation through viscosity reduction. However, additional C₂₋₃ alkanol can be added at any time during the process such as, for example, if most or all of the alkanol has been removed from the composition and it is deemed desirable to reduce the viscosity of the composition or suppress foam formation.

Use of at least one water miscible C₂₋₃ alkanol in the processes of this invention for producing a concentrated liquid composition of this invention provides still another advantage. In particular, because a C₂₋₃ alkanol forms an azeotrope with water, this enables water to be removed more efficiently by an evaporation procedure in order to form a more highly concentrated solution of dissolved potassium ibuprofen. Still further, because of the miscibility of the C₂₋₃ alkanols with water, the azeotrope of water and C₂₋₃ alkanol can be recycled to step a) and thereby provide more efficient utilization of the water miscible C₂₋₃ alkanol(s) used.

In step a) the components ibuprofen, potassium base, water, and at least one water miscible C₂₋₃ alkanol, can be brought together in various ways and sequences. For example these components can be charged to a vessel individually in any order, or any two or more of them can be individually charged concurrently, again in any sequence when also charging one or more other component(s). Alternatively, the charging can involve use of various preformed subcombinations. Thus, for example, the total amount of ibuprofen and potassium base can be provided by charging an appropriate amount of preformed potassium ibuprofen powder or particles instead of potassium base and ibuprofen. If desired, the total amount of ibuprofen and potassium base can be provided by charging some potassium base and/or ibuprofen before, at the same time, and/or after such preformed potassium ibuprofen is charged. Similarly, the water and the water miscible C₂₋₃ alkanol(s) can be premixed and added as a mixture, optionally with separate addition of more of either one or both of them. Still other ways of getting these components into the vessel exist and can be used. Although there is nothing critical about how the components are brought together in step a) of the process, it is preferable to add the potassium base and water incrementally, either separately or in combination, or both, to the ibuprofen while agitating the reaction mixture.

In conducting step a) of the process any potassium base that can form a potassium salt of ibuprofen can be used. However for pharmaceutical uses, the potassium base employed in the process must not contribute pharmaceutically unacceptable species such as cyanide. Non-limiting examples of suitable bases include for example potassium carbonate, potassium bicarbonate, potassium hydroxide, potassium silicate, potassium oxide, one or more water-soluble potassium salts of inorganic acids of phosphorus, and mixtures of any two or more of the foregoing. Use of potassium carbonate or potassium hydroxide or a combination of these as the base is preferred.

Typically the proportion of potassium base used in step a) will be such that the equivalent ratio of ibuprofen to the potassium base is in the range of about 0.9:1 to about 1:1, and preferably in the range of about 0.95:1 to about 0.98:1. It will of course be understood that in the reaction with ibuprofen, 1 equivalent of a potassium base having 1 atom of potassium per molecule (e.g., KOH) is 1 mole thereof. On the other hand, 1 equivalent of a potassium base having 2 atoms of potassium per molecule, such as potassium carbonate (K₂CO₃), is 0.5 mole thereof. It will be appreciated therefore that the potassium ibuprofen in the compositions of this invention has a mole ratio of potassium to ibuprofen in the range of about 0.9:1 to about 1:1 and preferably in the range of about 0.95:1 to about 0.98:1.

According to strict chemical theory, potassium 2-(4-isobutylphenyl)propionate is made up of 1 atom of potassium per molecule of ibuprofen which has been neutralized by the potassium base used. But if the reaction does not go to completion, there can be some unreacted potassium base or unreacted ibuprofen present in the potassium ibuprofen compositions of this invention. Moreover, there can be some free potassium base or some free ibuprofen present in the potassium ibuprofen compositions of this invention because of the use of the ibuprofen and the potassium base in an equivalent ratio specified in the immediately preceding paragraph in which one or the other such reactant is present in an excess over the precise potassium:ibuprofen stoichiometric ratio of 1:1. Therefore for convenience, when reference is made herein, including the claims, to the potassium ibuprofen in a composition of this invention having a mole ratio of potassium to ibuprofen in the range of, say, about 0.9:1 to about 1.05:1, this means that if a sample of the concentrated clear, or hazy, cloudy, or turbid, potassium ibuprofen liquid composition were to be analyzed, the analysis, if properly conducted, would indicate that the ratio of potassium to ibuprofen in the composition is in that specified range. The use of such ratio is not to be interpreted to refer to any change in the actual molecular structure of potassium 2-(4-isobutylphenyl)propionate when in solid form. Rather, the use of such ratio is to be understood to mean that the potassium 2-(4-isobutylphenyl)propionate is in whatever chemical form(s) it exists while in the liquid composition of this invention and that if all liquids were removed from the composition, the dry residue would contain potassium 2-(4-isobutylphenyl)propionate and optionally at least some free ibuprofen or some free potassium base.

In conducting step a) it is desirable to minimize the amount of water used. Thus, although the weight ratio of water to ibuprofen can be very large, e.g., 1:0.1 or more, the amount of water to be removed in step c) of the process would be unnecessarily large. Thus as a practical matter, one should employ an amount of water that does not require excessive water removal after completion of the reaction. In selecting the weight ratio of water to ibuprofen for use in any given situation, one should also take into consideration the water solubility of the potassium base being employed. Thus, when using such bases as potassium hydroxide and potassium oxide, which have higher water solubilities than, say, potassium bicarbonate, the amount of water used can be smaller than when using a less soluble base such as potassium bicarbonate. Generally speaking, it is convenient to use weight ratios of water to ibuprofen in the range of about 0.2:1 to about 0.8:1 and preferably in the range of about 0.25:1 to about 0.40:1.

Step a) of the process is typically conducted at ambient room temperature but can be conducted at a reduced temperature (e.g., down to about 10° C.), or at an elevated temperature (e.g., up to about 60° C.), if desired. In short, any temperature at which the components can be mixed without undue difficulty can be employed in step a).

In step b) of the process, the mixture is heated under conditions that do not result in visually-observable color formation in the product composition being formed. The temperature to which the mixture is heated can be anywhere within the range of from above room temperature up to about 80° C. Since the time of the operation is inversely proportional to the temperature, it is desirable to heat the mixture to a temperature of at least about 40° C. although the operation can be conducted at temperatures between about room temperature and about 40° C., for longer periods of time. Thus, as long as the temperature does not exceed about 80° C., the temperature at which the operation is conducted is primarily a matter of choice.

In conducting step b) exposure to free oxygen is kept to a minimum, especially when operating at temperatures in the range of about 70 to about 80° C. Thus it is desirable to operate under an inert atmosphere such as nitrogen, argon, neon, krypton, or the like, or to operate under partial vacuum. Even at lower temperatures, e.g., in the range of about 40 to about 70° C. it is prudent to operate in a closed system having a relatively small head space, under an inert atmosphere, or under a partial vacuum, but this is not essential so long as color development in the composition does not occur. It can be seen, therefore, that step b) is desirably conducted in a substantially oxygen-free environment. By “substantially oxygen-free” is meant that the system contains either no free oxygen or contains an amount of free oxygen that does not result in visually-observable color formation in the product composition being formed. While on the subject of conditions used in step b), it is worth noting that when employing potassium carbonate (or any other less soluble potassium base) and employing water to potassium carbonate weight ratios of less than 1, it is desirable to use temperatures in the range of about 60 to about 80° C. to facilitate dissolution of the potassium carbonate in the relatively small quantity of aqueous solvent medium employed.

In any or all of steps a), b), and c) above, an azeotropic solvent can be added so that on concentrating the resulting composition in step c) to form a concentrated liquid composition devoid of such azeotropic solvent. Non-limiting examples of suitable azeotropic solvents include toluene, n-hexane, n-heptane, ethanol, ethylbenzene, ethyl acetate, or the like. At present, toluene and n-hexane are the preferred azeotropic solvents.

To effect concentration of the composition in step c), all or a portion of the composition formed in b) is subjected to flashing or distillation to remove some water and water miscible C₂₋₃ alkanol(s) from the composition. Evaporation of these solvents can be conducted using conventional equipment such as a wiped film evaporator or spray dryer. The concentration operation of step c) is typically conducted at reduced pressure and at elevated temperatures sufficient to remove some of the water and the water miscible C₂₋₃ alkanol(s) and thereby produce the concentrated pourable potassium ibuprofen liquid composition containing at least about 60 wt % of dissolved potassium ibuprofen and as high as about 90 wt % of dissolved potassium ibuprofen in a liquid medium composed chiefly of water together with at least one water miscible C₂₋₃ alkanol. In many cases the pourable compositions of this invention formed in c) are clear and homogeneous. However, in some cases the pourable compositions of this invention formed in c) are turbid especially when the liquid is maintained at or below ambient room temperature. By “turbid” is meant that the composition is hazy or cloudy in appearance and may contain particles or sediment that can be removed by ordinary physical solids/liquid separation techniques such as centrifugation or decantation. As between the pourable clear homogeneous compositions of this invention and the pourable turbid compositions of this invention, the pourable clear homogeneous compositions of this invention are preferred. If desired, it is often possible to convert a pourable turbid composition of this invention into a pourable clear homogeneous composition of this invention by use of ordinary physical solids/liquid separation techniques such as centrifugation or decantation or by adjusting the alkanol to water ratio of the composition.

Another way of preparing the concentrated liquid compositions of this invention is to procure or preform the potassium ibuprofen for use as a starting material. Such potassium ibuprofen may contain excess unreacted ibuprofen or unreacted potassium base but should have a mole ratio of ibuprofen moiety to potassium in the range of about 0.9:1 to about 1:1, preferably in the range of about 0.95:1 to about 0.98:1. In this mode of operation, the potassium ibuprofen is mixed with water and an excess amount of at least one water miscible C₂₋₃ alkanol, and the resultant mixture is heated, preferably with agitation, to evaporate by flashing, distillation, vacuum distillation, or the like, an alkanol-water mixture and thereby form in one or more stages a composition of this invention. In this mode of operation use of an azeotropic solvent is unnecessary.

If desired, small amounts (e.g., up to about 5 wt %) of pharmaceutically-acceptable excipients, preservatives, sweeteners, or the like can be included in the compositions of this invention. Examples of such materials include glycerol, methylparaben, and sorbitol. Preferably, however, the compositions of this invention are prepared from nothing other than water, at least one water miscible C₂₋₃ alkanol, (more preferably ethanol only), at least one polyoxyethylene glycol, ibuprofen, and potassium base (or preformed potassium ibuprofen as a partial or total replacement for ibuprofen and potassium base). Thus especially preferred compositions of this invention contain in addition to potassium ibuprofen (in whatever chemical form or forms it exists while in a concentrated pourable potassium ibuprofen liquid composition of this invention), only water, at least one polyoxyethylene glycol, optionally at least one C₂₋₃ alkanol, and optionally unreacted ibuprofen, and possibly impurities that may be in the materials used to prepare the compositions of this invention and/or that result from the manufacturing operations employed in forming the compositions of this invention.

Methods of providing concentrated pourable potassium ibuprofen liquid compositions in individual pharmaceutical dosage forms constitute still additional embodiments of this invention. In general, such methods comprise encapsulating individual pharmaceutical dosage portions of such composition within gelatin shells or soft shells made from other suitable substances such as a composition comprised of a modified starch and iota-carrageenan. This can be accomplished by various techniques such as encapsulation techniques of types such as described for example in U.S. Pat. Nos. 2,234,479; 5,209,978; 6,340,473; 6,569,363; 6,589,536; Published International Application WO 98/42294 published Oct. 1, 1998; The Theory and Practice of Industrial Pharmacy, Leon Lachman, Herbert A. Lieberman and Joseph L. Kanig, editors, 3rd Edition, 1986, Lea & Febiger, Philadelphia, Pa., Publishers, and Ebert, Soft Elastic Gelatin Capsules: A Unique Dosage Form, Pharmaceutical Technology, October 1977. The encapsulation techniques described in such references are incorporated herein by reference as descriptive of techniques that can be utilized to form individual pharmaceutical dosage forms of this invention. Thus use can be made of rotary die encapsulation processes, reciprocating die encapsulation processes, concentric cylinder processes, and film-enrobing processes all of which are known in the art. Of such encapsulation processes, the rotary die process is presently preferred for use in the practice of this embodiment of the invention.

Machinery for producing soft capsules of the type herein involved, is available from various manufacturers. For example, one may utilize model VSG-172A Softgel manufacturing line produced by Vanguard Pharmaceutical Machinery, Inc., USA which includes an advanced design rotary die encapsulator as well as various associated equipment. Other commercially available equipment for producing soft capsules are models CS-M3 and Model CS-J1 Soft Gel machines available from Daesung Corporation, Daesung B/D 3F, 9-1, Yangpyong 1-dong, Youngdeungpo-ku, Seoul, 150-101 Korea; and Models SGM-1000 and SGM-2000 machines available from Technophar Equipment and Service Limited, 1370 Argyl Road, Windsor, Ontario, N8Y 3K7, Canada.

Thus there is provided pursuant to an embodiment of this invention, a method of providing potassium ibuprofen in individual pharmaceutical dosage forms, which method comprises encapsulating individual pharmaceutical dosage portions of a concentrated pourable potassium ibuprofen liquid composition of this invention within gelatin shells. Preferably in conducting this method the gelatin shells are formed by sealing ribbons of gelatin together around said individual dosage portions of the concentrated pourable potassium ibuprofen liquid composition to thereby encapsulate said dosage portions. More preferably the gelatin shells formed in these methods are soft gelatin shells. In particularly preferred embodiments, the foregoing methods are practiced using a concentrated pourable potassium ibuprofen liquid composition of this invention wherein the dissolved potassium ibuprofen in said composition has an analyzable mole ratio of potassium to ibuprofen in the range of about 0.95:1 to about 0.98:1, and especially wherein the amount of dissolved potassium ibuprofen in such composition is in the range of about 80 to about 90 wt %, and the ethanol and the water are in a weight ratio of ethanol to water in the range of about 0.30:1 to about 0.80:1.

Also provided by this invention is an article of manufacture which comprises at least one pharmaceutical capsule defining an interior enclosed space, said capsule being sized and shaped for oral administration, and containing within said space a quantity of a concentrated pourable potassium ibuprofen liquid composition comprising (i) potassium ibuprofen in dissolved form, (ii) water; and (iii) at least one polyoxyethylene glycol, optionally ethanol of pharmaceutically acceptable purity, and optionally ibuprofen in free acid form, wherein the composition contains an amount of dissolved potassium ibuprofen in the range of about 60 to about 90 wt %, wherein the total wt % of (i), (ii), and (iii) in the composition is at least about 95 wt %, and wherein the composition is pourable at about 25° C. Preferably such article is a soft gelatin capsule, especially a seamless soft gelatin capsule, or a soft capsule that comprises a modified starch and iota-carrageenan (see for example U.S. Pat. No. 6,340,473. It these articles the dissolved potassium ibuprofen in the encapsulated composition preferably has an analyzable mole ratio of potassium to ibuprofen in the range of about 0.95:1 to about 0.98:1, especially wherein the amount of dissolved potassium ibuprofen in such composition is in the range of about 80 to about 90 wt %, and wherein the ethanol and the water are in a weight ratio of ethanol to water in the range of about 0.30:1 to about 0.80:1.

The term “analyzable mole ratio of potassium to ibuprofen” as used herein including the claims means that if the liquid composition being referred to is subjected to analysis, the results of the analysis will indicate that the composition has a mole ratio of potassium to ibuprofen that is in the range specified. This term does not mean that the composition must be analyzed; rather it means only that if one elects to analyze the composition, the analytical results will indicate that the range of mole ratios specified has been complied with. Similarly this term does not constitute, by implication or otherwise, a description of the chemical form in which the potassium ibuprofen exists while dissolved in the composition. As pointed out hereinabove, the potassium ibuprofen can be in whatever chemical form or forms it exists during the time it remains dissolved in the composition.

Another embodiment of this invention is a pharmaceutical product formed from at least one component comprised of a concentrated liquid composition according this invention. The pharmaceutical products of this invention may be in liquid, solid, or a combination of liquid and solid form (e.g., gel encapsulation). Still another embodiment of this invention is a process of administering, to a mammal exhibiting at least one symptom responsive to analgesic treatment (e.g., pain, fever, swelling, etc.), a pharmaceutically effective amount of a pharmaceutical product of this invention. The pharmaceutical product of this invention may include additional components typically present in conventional liquid, solid and/or encapsulated pharmaceutical products. Suitable non-limiting examples of such other components include viscosity modifiers, flavoring, sweeteners, colorants, stabilizers or other preservatives and the like. The product is typically administered orally. When administering the product, the pharmaceutically effective amount employed may vary, but should be sufficient to trigger a discernable analgesic symptomatic response. In most cases, the pharmaceutically effective amount will be an amount sufficient to provide to the mammal an in situ amount of an acid form of ibuprofen in the range of about 20 to about 800 mg, since the potassium ibuprofen will normally convert to the acid form after administration.

Another aspect of this invention relates to methods of utilizing the concentrated liquid compositions of this invention as raw materials for producing less concentrated liquid compositions for use in forming filled capsules and other pharmaceutically-acceptable liquid dosage forms such as syrups, suspensions, elixirs, and the like. This aspect is of particular advantage in that commercial pharmaceutical manufacturers that are accustomed to using more dilute liquid pharmaceutical compositions in their operations, can easily utilize the concentrates of this invention to form more dilute liquid compositions for their use. Thus, still another embodiment of this invention is a method of preparing a diluted formulation from any concentrated pourable liquid composition this invention described and/or claimed herein, which method comprises mixing such concentrated pourable liquid composition with (i) a solution of at least one pharmaceutically active ingredient in at least one pharmaceutically-acceptable solvent, or (ii) a combination of (a) at least one pharmaceutically active ingredient and (b) at least one pharmaceutically-acceptable solvent, (a) and (b) being mixed separately with the concentrated pourable liquid composition, or (iii) at least one pharmaceutically-acceptable excipient, or (iv) a combination of any two or all three of (i), (ii), and (iii), to form a less concentrated homogeneous solution which is stable over a temperature range of about 0° C. to about 50° C. Such formulation is useful for preparing various liquid dosage forms of ibuprofen, and is particularly adapted for use in filling soft gelatin capsules.

Any known pharmaceutically active ingredient can be utilized in forming solutions to be used in the method described in the immediately preceding paragraph, provided such active ingredient is compatible with ibuprofen and results in a composition which is acceptable for pharmacological use. Similarly, any known pharmaceutically-acceptable excipient can be utilized in the above method provided such excipient is compatible with ibuprofen and results in a composition which is acceptable for pharmacological use. Among suitable excipients are suitable solvents, including, for example, glycerin, propylene glycol, and polyvinylpyrrolidone, and combinations thereof. Preferred solvents are (i) water, (ii) at least one C₂₋₃ alkanol, (iii) at least one polyethylene glycol having a number average molecular weight in the range of about 200-2000 Daltons or (iv) a combination of any two or all three of (i), (ii), and (iii).

Additional ingredients which enhance the solubility of the active pharmaceutical ingredient in polyethylene glycol can be used as well, provided such ingredients are present only in amounts sufficient to preserve the desired viscosity and that do not degrade gelatin capsules. Examples of additional ingredients include, but are not limited to, glycerin, propylene glycol, and polyvinylpyrrolidone, and combinations thereof. The amount and combination of additional ingredient(s) used will vary according to the chemical properties of the other ingredients used in the process. Preferred pharmaceutically-acceptable excipients include acidic excipients, as inclusion of such excipients in the more dilute compositions reduces the pH and thereby minimizes the likelihood of premature deterioration of gelatin capsules. Non-limiting examples of suitable acidic excipients include citric acid, tartaric acid, and the like. Other conventional excipients can be used in conjunction with the method of this invention for preparing a diluted formulation. These include, but are not limited to, preservatives, stabilizers, wetting agents, coloring agents, and the like.

A preferred embodiment of this aspect of the invention is a method of preparing a diluted formulation from a concentrated pourable liquid composition of this invention described and/or claimed herein, which method comprises diluting such concentrated pourable liquid composition with at least one of a) and b) which are as follows:
a) a more dilute solution preformed from ibuprofen and at least one pharmaceutically-acceptable solvent, said more dilute solution being more dilute than said concentrated pourable liquid composition; or
b) separate additions to said concentrated pourable liquid composition of diluting quantities of ibuprofen and of at least one pharmaceutically-acceptable solvent.
   The preferred pharmaceutically-acceptable solvent of a) orb) is (i) water, (ii) at least one C₂₋₃ alkanol, (iii) at least one polyethylene glycol having a number average molecular weight of at least about 200 Daltons that is in the liquid state at about 20° C., or (iv) a combination of any two or all three of (i), (ii), or (iii).

The practice and advantages of this invention are illustrated by the following examples in which all percentages are by weight.

The general procedure used in the Examples involved preparing initial concentrated solutions for use as raw materials which in turn were used in preparing various compositions of this invention. These initial concentrated solutions were highly concentrated clear stable liquid formulations consisting of only potassium ibuprofen, water and ethanol. In all cases, stoichiometric amounts of ibuprofen and potassium carbonate were used as starting materials, i.e., two moles of ibuprofen and one mole of potassium carbonate were used.

More particularly, ibuprofen, manufactured by Albemarle Corporation, with a medium particle size of 40 micrometers was used as a starting raw material. The ibuprofen was first de-lumped by sieving the ibuprofen through a No. 12 stainless steel sieve. Potassium carbonate, anhydrous (138.2 grams) was dissolved in 150 grams of de-ionized water in a 2-liter flask sitting on a magnetic stirrer. A 2-inch magnetic stirring bar was added to the flask to provide agitation. The ibuprofen, 412 grams, was added to the flask through a powder funnel. Anhydrous ethanol (300 grams) was incrementally added to the flask under agitation. After completion of the addition, agitation continued until all foams due to carbon dioxide evolution subsided. The content of the flask was then quantitatively transferred into a 2-liter round-bottom flask suitable for rotary evaporator operation. The flask was then connected to a Rotavapor RE11 (manufactured by Buchi) rotary evaporator and heated to 70° C. under rotation. After the contents of the flask became clear, vacuum provided by an aspirator pump was used to evaporate part of the volatiles (carbon dioxide, water and ethanol). After about 300 to 320 grams of the solvents were removed, the evaporation operation was momentary stopped. After an additional 300 grams of ethanol was added to the flask, the rotary evaporator solvent removal process was resumed. The process was completed after another about 320 grams of solvent was removed. A clear potassium ibuprofen liquid composition, weighing about 600 grams, was obtained from several such runs.

Typically, a liquid potassium ibuprofen lot is prepared by mixing materials collected from three of the above-mentioned runs. A sample of the lots was submitted for NMR analyses. The compositions of four of the lots as determined by NMR are given in Table 1. All of these lots are highly concentrated stable liquid potassium ibuprofen compositions. These lots were then used to prepare and study compositions of this invention containing varying amounts of added ibuprofen and polyethylene glycol.

TABLE 1
Composition of Potassium Ibuprofen Lots

| Lot I.D. | Potassium Ibuprofen, wt % | Water, wt % | Ethanol, wt % |
|---|---|---|---|
| S-3 | 84.68 | 10.72 | 4.59 |
| S-4 | 85.76 | 10.33 | 3.91 |
| S-5 | 85.33 | 10.54 | 4.13 |
| S-7 | 86.10 | 9.50 | 4.41 |

### EXAMPLE 1

30 Grams of PEG 400 and 507 grams of potassium ibuprofen Lot S-5 were added to a 2-liter round-bottom flask. The evaporation flask was then installed in the rotary evaporator and heated to about 70° C. under rotation. After the contents of the flask became clear, vacuum provided by an aspirator pump was used to remove part of the solvents (water and ethanol) through evaporation. After 12 grams of solvent was evaporated, a highly concentrate stable clear liquid of this invention was obtained composed of 81.7 wt % potassium ibuprofen, 9.7 wt % water, 2.8 wt % ethanol and 5.7 wt % polyethylene glycol (PEG 400), all as determined through use of NMR analyses.

### EXAMPLE 2

The process described in Example 2 was repeated except the amount of volatiles removed was increased from 12 grams to 21 grams. After the 21 grams of solvent had been removed by evaporation, the flask contained a highly concentrated stable clear liquid of this invention. This composition was composed of 85.2 wt % potassium ibuprofen, 7.9 wt % water, 0.9 wt % ethanol and 6 wt % PEG 400, all as determined through use of NMR analyses.

### EXAMPLE 3

638 Grams of potassium ibuprofen Lot S-5, 17.8 grams of ibuprofen, and 41.2 grams of PEG 400 was added to a 2-liter round-bottom flask. The evaporation flask was then installed in the rotary evaporator and heated to about 70° C. under rotation. After the contents of the flask become clear, vacuum provided by an aspirator pump was used to evaporate part of the solvents (water and ethanol). After 24 grams of solvent had been removed through evaporation, a highly concentrated stable clear liquid composition of this invention remained in the flask. This composition was composed of 2.7 wt % ibuprofen, 80.8 wt % potassium ibuprofen (KIBU), 8.8 wt % water, 1.9 wt % ethanol and 6.3% PEG 400, all as determined through use of NMR analyses.

### EXAMPLE 4

800 Grams of potassium ibuprofen Lot S-4 and 55 grams of PEG 400 were added to a 2-liter round-bottom flask. The evaporation flask was then installed in the rotary evaporator and heated to about 70° C. under rotation. After the contents of the flask became clear, vacuum provided by an aspirator pump was used to evaporate part of the solvents (water and ethanol). After 35 grams of solvent had been removed through evaporation, the flask contained a highly concentrated stable clear liquid composition of this invention. NMR analyses indicated that the composition was composed of 83.7 wt % potassium ibuprofen, 8.5 wt % water, 1.3 wt % ethanol and 6.7 wt % PEG 400.

### EXAMPLE 5

To a 2-liter round-bottom evaporation flask were added 500 grams of potassium ibuprofen from lot S-3, 62.5 grams of PEG 400 and 27 grams of ibuprofen. The flask was then installed in the rotary evaporator and then heated to about 70° C. under rotation. After the contents of the flask became clear, vacuum provided by an aspirator pump was used to evaporate part of the solvents (water and ethanol). After about 41 grams of solvent had been removed, the contents of the flask were poured into a bottle for storage. The capped bottle contents were allowed to cool down to ambient room temperature and stored. After a few days of storage at room temperature the contents of the bottle existed as a cloudy liquid. This composition was indicated by NMR to be composed of 4.9 wt % ibuprofen, 77.3 wt % potassium ibuprofen, 6.6 wt % water, 0.4 wt % ethanol and 11.2 wt % PEG 400.

### EXAMPLE 6

10 Grams of PEG 400 was added to the composition formed in Example 5. The composition became clear upon being heated to 60° C. with agitation. Upon cooling down to ambient room temperature conditions, and after a few days at room temperature the composition turned cloudy. NMR analysis indicated this composition contained 4.8 wt % ibuprofen, 75.9 wt % potassium ibuprofen, 6.0 wt % water, 0.3 wt % ethanol and 13 wt % PEG 400, turns cloudy.

### EXAMPLE 7

40 Grams of PEG 400 and 799 grams of potassium ibuprofen Lot S-7 were added to a 2-liter round-bottom flask. The evaporation flask was then installed to the rotary evaporator and heated to about 70° C. under rotation. After the contents of the flask became clear, vacuum provided by an aspirator pump was used to remove part of the solvents (water and ethanol) through evaporation. After 35 grams of solvent had been evaporated, the solvent removal was momentary stopped, 10 grams of PEG 400 were added to the flask, and the contents were mixed together. After storage at ambient room temperature for a few days this composition was found to have undergone some precipitate formation. NMR analysis indicated that the liquid phase was composed of 84.5 wt % potassium ibuprofen 9.4 wt % water, 1.41 wt % ethanol and 6.14 wt % PEG 400,

### EXAMPLE 8

Added to a round-bottom flask were 250 grams of the liquid composition formed in Example 6 which consisted of 4.84 wt % ibuprofen, 75.9 wt % potassium ibuprofen, 6.0 wt % water, 0.31 wt % ethanol and 13.0 wt % PEG 400, and 750 grams of the liquid phase product formed in Example 7 which consisted of 84.5 wt % potassium ibuprofen, 9.44 wt % water, 1.41 wt % ethanol and 4.74 wt % PEG 400, 10 grams of water and 20 grams of PEG 400. The flask was heated to around 60° C. with occasional shaking until the composition became clear. The system remained clear and stable after cooling. The resultant highly concentrated stable clear liquid composition of this invention was indicated by NMR to contain 1.2 wt % ibuprofen, 80.0 wt % potassium ibuprofen, 9.3 wt % water, 1.1 wt % ethanol and 8.5 wt % PEG 400.

### EXAMPLE 9

800 Grams of potassium ibuprofen Lot S-7, 65 grams of PEG 400 and 17 grams of ibuprofen were added to a 2-liter round-bottom flask. The flask is then installed in the rotary evaporator and heated to about 70° C. under rotation. After the contents of the flask became clear, vacuum provided by an aspirator pump was used to evaporate part of the solvents (water and ethanol). 30 Grams of solvent were evaporated leaving a highly concentrated stable clear liquid composition of this invention, which per NMR analysis was composed of 2.0 wt % ibuprofen, 80.8 wt % KIBU, 7.8 wt % water, 2.2 wt % ethanol and 7.5 wt % PEG 400.

### EXAMPLE 10

638 Grams of potassium ibuprofen Lot S-5, 17.8 grams of ibuprofen, and 41.2 grams of PEG 400 were added to a 2-liter round-bottom flask. The evaporation flask was then installed in the rotary evaporator and heated to about 70° C. under rotation. After the contents of the flask became clear, vacuum provided by an aspirator pump was used to evaporate part of the solvents (water and ethanol). After 24 grams of solvent were evaporated, an additional 18 grams of PEG 400 was added to the flask and the evaporation was continued until another 20 grams of solvent had been removed. The composition was indicated by NMR to be composed of 2.7 wt % ibuprofen, 80.6 wt % potassium ibuprofen, 7 wt % water, 0.75 wt % ethanol and 8.8 wt % PEG 400. It was found that the composition became turbid after storage in an ambient room temperature environment for a few days.

### EXAMPLE 11

Product formed in Example 10 was added to a 2-liter flask along with additional water and ethanol. The contents of the flask were then heated to around 60° C. with mixing until the contents of the flask became clear. Upon cooling back to ambient room temperature, in a few days, the contents of the flask turned cloudy. This composition was indicated by NMR to be composed of 2.6 wt % ibuprofen, 78.7 wt % KIBU, 8.5 wt % water, 1.8 wt % ethanol and 8.5 wt % PEG 400.

Table 2 summarizes the analytical data of the compositions of this invention formed as in Examples 1-11.

TABLE 2

| | IBU | KIBU | Water | Ethanol | PEG 400 |
|---|---|---|---|---|---|
| Example 1 | 0 | 81.7 | 9.7 | 2.8 | 5.7 |
| Example 2 | 0 | 85.2 | 7.9 | 0.9 | 6 |
| Example 3 | 2.7 | 80.8 | 8.8 | 1.9 | 6.3 |
| Example 4 | 0 | 83.7 | 8.5 | 1.3 | 6.7 |
| Example 5 | 4.9 | 77.3 | 6.6 | 0.4 | 11.2 |
| Example 6 | 4.8 | 75.9 | 6.0 | 0.3 | 13 |
| Example 7 | 0 | 84.5 | 9.4 | 1.4 | 6.1 |
| Example 8 | 1.2 | 80.0 | 9.3 | 1.1 | 8.5 |
| Example 9 | 2 | 80.8 | 7.8 | 2.2 | 7.5 |
| Example 10 | 2.7 | 80.6 | 7 | 0.75 | 8.8 |
| Example 11 | 2.6 | 78.7 | 8.5 | 1.8 | 8.5 |

It can be seen from Examples 5-8 that when a composition of this invention is formed in which some turbidity or minor precipitate formation occurs, the composition can be transformed into a highly stable composition of this invention by use of suitable blending techniques. Generally speaking, if the cloudy or turbid composition has a relatively high concentration of free ibuprofen (i.e., ibuprofen in its acid form) addition to the composition of more polyoxyethylene glycol and/or C₂₋₃ alkanol will result in a concentrated solution of this invention having improved storage stability. Conversely, if the cloudy or turbid composition contains either no free ibuprofen or a relatively low concentration of free ibuprofen, addition to the composition of more water will result in a concentrated solution of this invention having improved storage stability. Such additions of either polyoxyethylene glycol and/or C₂₋₃ alkanol or water should be of small quantities so that the resultant concentrate is not overly diluted.

Tables 3 and 4 set forth illustrative preferred solvent systems for use with potassium ibuprofen which optionally contains up to about 3 wt % of ibuprofen in its free acid form. The solvent systems of Table 3 are recommended for use with compositions containing about 60 wt % of potassium ibuprofen whereas the solvent systems of Table 4 are recommended for use with compositions containing about 70 wt % of potassium ibuprofen. Linear interpolations can be used between the values of Tables 3 and 4 for systems in which the content of potassium ibuprofen is between 60 and 70 wt %. Similarly, linear interpolations can be used between the values of Tables 4 and 2 for systems in which the content of potassium ibuprofen is between 70 and about 80-85 wt %.

TABLE 3
SOLVENT SYSTEMS FOR COMPOSITIONS CONTAINING ABOUT
60 WT % POTASSIUM IBUPROFEN AND OPTIONALLY UP TO
ABOUT 3 WT % OF IBUPROFEN IN THE FREE ACID FORM

| | | | | Total |
|---|---|---|---|---|
| | Water, wt % | Alkanol, wt % | PEG, wt % | Solvent, wt % |
| Example 1 | 21.3 | 6.2 | 12.6 | 40.1 |
| Example 2 | 21.3 | 2.4 | 16.2 | 39.9 |
| Example 3 | 20.7 | 4.5 | 14.8 | 40 |
| Example 4 | 20.5 | 3.1 | 16.4 | 40 |
| Example 8 | 19.8 | 2.3 | 17.9 | 40 |
| Example 9 | 17.7 | 5.1 | 17.2 | 40 |

TABLE 4
SOLVENT SYSTEMS FOR COMPOSITIONS CONTAINING ABOUT
70 WT % POTASSIUM IBUPROFEN AND OPTIONALLY UP TO
ABOUT 3 WT % OF IBUPROFEN IN THE FREE ACID FORM

| | | | | Total |
|---|---|---|---|---|
| | Water, wt % | Alkanol, wt % | PEG, wt % | Solvent, wt % |
| Example 1 | 16.2 | 4.6 | 9.2 | 30 |
| Example 2 | 16.0 | 1.8 | 12.2 | 30 |
| Example 3 | 15.5 | 3.4 | 11.1 | 30 |
| Example 4 | 15.4 | 2.4 | 12.2 | 30 |
| Example 8 | 14.8 | 1.7 | 13.4 | 29.9 |
| Example 9 | 13.3 | 3.8 | 12.9 | 30 |

In the foregoing Examples numerical values concerning the compositions of the products obtained are based on NMR analyses and thus subject to deviations inherent in use of such procedures. Also the actual experiments on which these Examples are based were conducted for convenience using anhydrous ethyl alcohol from J. T. Baker Inc., a division of Mallinckrodt Baker, Inc. The label of this ethyl alcohol indicates that the product is denatured with 5.3% (v/v) isopropyl alcohol and that the product was made from Specially Denatured Alcohol 3A which consists of 5 volumes of methanol and 100 volumes of 200 proof ethanol. Use of such denatured alcohol was deemed entirely suitable for conducting the laboratory experiments in connection with this invention. When employing anhydrous ethanol in actual commercial practice in connection with product to be used for human internal consumption, one should use a purer grade of ethyl alcohol such as pure 200 proof ethyl alcohol which contains only trace amounts, if any, of any other alcohol.

Components referred to herein by chemical name or formula, whether referred to in the singular or plural, are identified as they exist prior to coming into contact with another substance referred to by chemical name or chemical type (e.g., another component or a solvent). Also, even though the claims hereinafter may refer to substances, components and/or ingredients in the present tense (e.g., “comprises” or “is”), the reference is to the substance, component or ingredient as it existed at the time just before it was first contacted, blended or mixed with one or more other substances, components and/or ingredients in accordance with the present disclosure.

Except as may be expressly otherwise indicated, the article “a” or “an” if and as used herein is not intended to limit, and should not be construed as limiting, a claim to a single element to which the article refers. Rather, the article “a” or “an” if and as used herein is intended to cover one or more such elements, unless the text expressly indicates otherwise. This invention is susceptible to considerable variation within the spirit and scope of the appended claims.

1. A process of producing a concentrated potassium ibuprofen composition, which process comprises:
a) forming a mixture of ibuprofen, potassium base, water, and optionally at least one C₂₋₃ alkanol, the ratio of equivalents of potassium base to ibuprofen used being in the range of 0.9:1 to 1:1;
b) partially removing alkanol when used and water from mixture from a) thereby forming a product mixture enriched in dissolved potassium ibuprofen but not devoid of alkanol when used and water;
c) either continuing or discontinuing removal of alkanol when used and water as in b), and mixing at least one polyethylene glycol with product mixture from b) to form a pourable polyethylene glycol-containing composition; and
d) if removal of alkanol when used and water is discontinued in c), optionally resuming removal of alkanol when used and water, in this case from pourable polyethylene glycol-containing composition from c) to thereby form a more concentrated pourable potassium ibuprofen liquid composition comprised of (i) potassium ibuprofen; (ii) water; (iii) at least one polyalkylene glycol; (iv) optionally at least one C₂₋₃ alkanol; and (v) optionally ibuprofen in free acid form; and
e) using amounts of ibuprofen, potassium base, water, polyethylene glycol(s) and optionally C₂₋₃ alkanol(s) in the process and removing amounts of such alkanol(s) when used and water in the process that provide a composition containing at least 60 wt % of potassium ibuprofen in dissolved form.

2. A process as in claim 1 which comprises:
a) forming a mixture of ibuprofen, potassium base, water, and at least one C₂₋₃ alkanol, the ratio of equivalents of potassium base to ibuprofen used being in the range of 0.9:1 to 1:1;
b) partially removing alkanol and water from mixture from a) thereby forming a product mixture enriched in dissolved potassium ibuprofen but not devoid of alkanol and water;
c) discontinuing removal as in b), and mixing at least one polyethylene glycol with product mixture from b) to form a pourable polyethylene glycol-containing composition; and
d) resume removal of alkanol and water, in this case from pourable polyethylene glycol-containing composition from c) to thereby form a more concentrated pourable potassium ibuprofen liquid composition comprised of (i) potassium ibuprofen; (ii) water; (iii) at least one polyalkylene glycol; (iv) optionally at least one C₂₋₃ alkanol; and (v) optionally ibuprofen in free acid form; and
e) using respective amounts of ibuprofen, potassium base, water, polyethylene glycol(s) and alkanol(s) in the process and removing respective amounts of alkanol(s) and water in the process that provide a composition containing at least 60 wt % of potassium ibuprofen in dissolved form.

3-4. (canceled)

5. A process as in claim 1 wherein the amounts used pursuant to e) and the amounts removed pursuant to e) provide a composition that also contains in the range of 5 to 10 wt % of water; in the range of more than 1 wt % but less than 10 wt % of polyethylene glycol(s); optionally up to 5 wt % of at least one C₂₋₃ alkanol; and (v) optionally up to 2.3 wt % of ibuprofen in free acid form.

6. (canceled)

7. A process as in claim 2 wherein the amounts used pursuant to e) and the amounts removed pursuant to e) provide
a composition that also contains in the range of 7.5 to 10 wt % of water;
a composition in which the weight ratio of (ii):(iii) in the composition is at least 1:1; and/or
a composition which contains (iv), optionally in an amount of which is not greater than 3 wt %.

8-12. (canceled)

13. A process as in claim 2 wherein (iv) is ethanol and wherein the amounts used pursuant to e) and the amounts removed pursuant to e) provide a composition in which the weight ratio of water+ethanol:polyethylene glycol in said composition is in the range of 1.4 to 2.4, or provide a composition which contains (v), optionally in an amount which is not greater than 2 wt %, or provide a composition which contains (iv) and (v), optionally wherein the amount of (iv) provided in said composition by the process is not greater than 3 wt %, and optionally wherein the amount of (v) provided in said composition by the process is not greater than 2.3 wt %.

14-18. (canceled)

19. A process as in claim 2 wherein (iv) is ethanol.

20-28. (canceled)

29. A process as in claim 1 wherein the removals as in b) and d) are carried out using, independently, evaporation procedures in which at least vaporized water and vaporized C₂₋₃ alkanol are formed and removed from the mixture, optionally wherein the evaporation procedure used is selected from distillation, flashing, azeotropic distillation, and vacuum distillation procedures.

30. (canceled)

31. A process as in claim 1 wherein the at least one polyethylene glycol used in the process has a number average molecular weight in the range of 200 to 2000 Daltons, or wherein the at least one polyethylene glycol used in the process has a number average molecular weight of at least about 200 Daltons and is a liquid at least at a temperature as low as 20° C.

32-40. (canceled)

41. A process as in claim 1 wherein after forming the mixture of a) and before conducting the partial removal of water and alkanol as in b), the mixture formed in a) is heated, optionally with agitation, to assist in forming potassium ibuprofen in dissolved form, the temperature used being below a temperature at which the mixture acquires a yellowish hue.

42. A process as in claim 5 wherein the amounts of alkanol and water removed in b) and d), and the amount of polyethylene glycol(s) used in c) provide a composition containing at least 70 wt % of potassium ibuprofen in dissolved form.

43. (canceled)

44. A process as in claim 1 which comprises:
a) forming a mixture of ibuprofen, potassium base, water, and at least one C₂₋₃ alkanol, the ratio of equivalents of potassium base to ibuprofen used being in the range of 0.9:1 to 1:1;
b) partially removing alkanol and water from mixture from a) thereby forming a product mixture enriched in dissolved potassium ibuprofen but not devoid of alkanol and water;
c) while continuing removal as in b), mixing at least one polyethylene glycol with product mixture from b) to form a pourable polyethylene glycol-containing composition; and
d) after mixing as in c), continuing removal of alkanol and water, in this case from pourable polyethylene glycol-containing composition from c) to thereby form a more concentrated pourable potassium ibuprofen liquid composition comprised of (i) potassium ibuprofen; (ii) water; (iii) at least one polyalkylene glycol; (iv) optionally at least one C₂₋₃ alkanol; and (v) optionally ibuprofen in free acid form; and
e) using respective amounts of ibuprofen, potassium base, water, polyethylene glycol(s) and alkanol(s) in the process and removing respective amounts of alkanol(s) and water in the process that provide a composition containing at least 60 wt % of potassium ibuprofen in dissolved form.

45. A process as in claim 44 wherein the amounts used pursuant to e) and the amounts removed pursuant to e) provide a composition containing at least 70 wt % of potassium ibuprofen in dissolved form, optionally providing a composition that also contains in the range of 5 to 10 wt % of water; in the range of more than 1 wt % but less than 10 wt % of polyethylene glycol(s); optionally up to 5 wt % of at least one C₂₋₃ alkanol;
and (v) optionally up to 2.3 wt % of ibuprofen in free acid form.

46-47. (canceled)

48. A concentrated pourable liquid composition comprised of
A) (i) in the range of 60 to 88 wt % of potassium ibuprofen in dissolved form; (ii) in the range of 5 to 10 wt % of water; (iii) in the range of more than 1 wt % but less than 10 wt % of one or more polyethylene glycols, (iv) optionally up to 5 wt % of at least one C₂₋₃ alkanol; and (v) optionally up to 2.3 wt % of ibuprofen in free acid form; wherein the weight ratio of (ii):(iii) is at least 0.9:1; and wherein the composition is pourable at least at 25° C.; or
B) (i) in the range of 60 to 86 wt % of potassium ibuprofen in dissolved form in a solvent system comprised of (ii) water, (iii) at least one polyethylene glycol, and (iv) at least one C₂₋₃ alkanol; and (v) optionally, ibuprofen in free acid form.

49. A composition as in claim 48 wherein said composition contains in the range of 7.5 to 10 wt % of water, and/or wherein the weight ratio of (ii):(iii) is at least 1:1.

50. (canceled)

51. A composition as in claim 48 wherein (iv) is present in said composition, optionally wherein the amount of (iv) present in said composition is not greater than 3 wt %; or wherein (v) is present in said composition, optionally wherein the amount of (v) present in said composition is not greater than 2 wt %; or wherein (iv) and (v) are present in said composition, optionally wherein the amount of (iv) present in said composition is not greater than 3 wt % and wherein the amount of (v) present in said composition is not greater than 2 wt %.

52. (canceled)

53. A composition as in claim 51 wherein (iv) is present in said composition, and wherein (iv) is ethanol, optionally wherein the weight ratio of water+ethanol:polyethylene glycol in said composition is in the range of 1.4 to 2.4.

54-65. (canceled)

66. A composition as in claim 48 wherein the at least one polyethylene glycol used has a number average molecular weight in the range of 200-2000 Daltons, or wherein the at least one polyethylene glycol used has a number average molecular weight at least about 200 Daltons and is in the liquid state at 20° C.

67-69. (canceled)

70. A composition as in claim 48 wherein the composition is devoid of glycerine, propylene glycol, polyvinylpyrrolidone, and polyoxyethylene sorbitan fatty acid ester, and/or wherein said composition contains at least 70 wt % of dissolved potassium ibuprofen.

71-76. (canceled)

77. A composition as in claim 48 wherein except for optional presence of (iv) and/or (v), except for an excess amount of unreacted potassium base, and except for optional presence of trace amounts of one or more impurities and/or manufacturing by-products, said composition contains only (i), (ii), and (iii).

78. A composition as in claim 77 wherein (iv) is present in said composition but (v) is not present therein, so that (i), (ii), (iii), and (iv) are present in said composition, or wherein (v) is present in said composition but (iv) is not present therein, so that (i), (ii), (iii), and (v) are present in said composition, or wherein (iv) and (v) are present in said composition.

79-81. (canceled)

82. A composition as in claim 48 wherein said composition is a clear single phase liquid composition.

83. A composition as in claim 82 wherein said clear single phase liquid composition is stable for at least 400 hours after preparation when stored in a closed container in the absence of light and at a temperature 5° C.

84. A composition as in claim 82 wherein said composition contains at least 70 wt % of dissolved potassium ibuprofen.

85-91. (canceled)

92. A composition of claim 48 wherein said composition is as in B), and wherein
the polyethylene glycol(s) used therein has or have a number average molecular weight of at least about 20° Daltons and is or are in the liquid state at 20° C., and/or wherein the weight ratio of water:polyoxyethylene glycol(s):alkanol(s) is in the ranges of 3.5-8.8:2.0-7.7:1; or
the water, polyethylene glycol(s) and alkanol(s) are in a weight ratio of water:polyethylene glycol(s)+alkanol(s) in the range of 0.4 to 2.5; or
the composition is either devoid of ibuprofen in its free acid state or the composition contains ibuprofen in its free acid state in an amount of up to about 4 wt % and wherein the weight ratio of (ii):(iii) is in the range of about 1:1 to about 2:1 and the weight ratio of (ii):(iv) is in the range of about 3 to about 10, optionally wherein the polyethylene glycol(s) used therein has or have a number average molecular weight of at least about 200 Daltons and is or are in the liquid state at 20° C., and optionally wherein the weight ratio of water:polyoxyethylene glycol(s):alkanol(s) is in the ranges of 3.5-8.8:2.0-7.7:1.

93-101. (canceled)

102. A method of preparing an analgesic pharmaceutical product in individual pharmaceutical dosage forms, which method comprises using a concentrated liquid composition of claim 48 in the formulation or preparation of said analgesic pharmaceutical product, and encapsulating individual pharmaceutical dosage portions of said product within gelatin shells.

103. A method as in claim 102 wherein said gelatin shells are formed by sealing ribbons of gelatin together around said individual dosage portions of the analgesic pharmaceutical product.

104. (canceled)

105. A method as in claim 102 wherein the dissolved potassium ibuprofen in said concentrated liquid composition has, prior to use in the formulation or preparation of the pharmaceutical product, a mole ratio of potassium to ibuprofen in the range of about 0.95:1 to about 0.98:1.

106. An article of manufacture which comprises at least one pharmaceutical capsule defining an interior enclosed space, said capsule being sized and shaped for oral administration, and containing within said space a quantity of an analgesic pharmaceutical product formulated or prepared by use of a concentrated liquid composition of claim 48.

107-109. (canceled)

110. An article of manufacture as in claim 106 wherein the dissolved potassium ibuprofen in said concentrated liquid composition has, prior to use in the preparation or formulation of the pharmaceutical product, a mole ratio of potassium to ibuprofen in the range of about 0.9:1 to about 1:1.

111-112. (canceled)

113. A method which comprises administering to a mammal exhibiting at least one symptom responsive to analgesic treatment, at least one pharmaceutical capsule defining an interior enclosed space, said capsule being sized and shaped for oral administration, and containing within said space a quantity of an analgesic pharmaceutical product formulated or prepared by use of a concentrated liquid composition of claim 48.

114. A method of preparing a diluted formulation from a concentrated pourable liquid composition of claim 48, which method comprises diluting said concentrated pourable liquid composition with at least one of a) and b) which are as follows:
a) a more dilute solution preformed from ibuprofen and at least one pharmaceutically-acceptable solvent, said more dilute solution being more dilute than said concentrated pourable liquid composition; or
b) separate additions to said concentrated pourable liquid composition of diluting quantities of ibuprofen and of at least one pharmaceutically-acceptable solvent.

115. A method of claim 114 wherein said diluting is done with a more dilute solution of a), or wherein said diluting is done with separate additions as in b), optionally wherein said pharmaceutically-acceptable solvent of a) or b) is (i) water, (ii) at least one C₂₋₃ alkanol, (iii) at least one polyethylene glycol having a number average molecular weight of at least about 200 Daltons that is in the liquid state at 20° C., or (iv) a combination of any two or all three of (i), (ii), or (iii).

116-117. (canceled)
